(19) United States
(12) Patent Application Publication
(10) Pub. No.: US 2006/0009682 A1
(43) Pub. Date: Jan. 12, 2006

(54) In-vivo examination apparatus

(76) Inventors: Nobuyuki Nagasawa, Hino-shi (JP); Yoshihisa Tanikawa, Chuo-ku (JP); Yoshihiro Kawano, Hachioji-shi (JP); Yasunori Makara, Hino-shi (JP)

Correspondence Address: KENYON & KENYON, 1500 K STREET NW, SUITE 700, WASHINGTON, DC, 20005, US

(21) Appl. No.: 11/159,204

(22) Filed: Jun. 23, 2005

(30) Foreign Application Priority Data
Jul. 6, 2004 (JP) 2004-199498

(51) Int. Cl. A61B 1/06

(52) U.S. Cl. 600/171; 600/182; 600/146

(57) ABSTRACT

The invention provides an in-vivo examination apparatus that can examine a minute examination site in a living organism with a simple configuration. The invention provides an in-vivo examination apparatus comprising a light source; a flexible light-conveying member that transmits light from the light source to irradiate the light from an end face thereof onto an examination site and that receives return light returning from the examination site at the end face thereof to transmit the return light; a long thin insertion part in which the light-conveying member is disposed along the longitudinal direction thereof; and an optical detector that detects the return light from biological tissue, which is transmitted through the insertion part via the light-conveying member. The end face of the insertion part, where the end face of the light-conveying member is exposed, is configured so as to be cut at an angle with respect to the longitudinal direction to provide a pointed portion that can incise the biological tissue at the tip thereof.

### BACKGROUND OF THE INVENTION

1. Field of the Invention

The present invention relates to an in-vivo examination apparatus for in-vivo examination of a living organism.

2. Description of Related Art

One conventionally known apparatus for examining the condition of tissue inside a living organism is the medical manipulator apparatus disclosed in Japanese Unexamined Patent Application Publication No. HEI 8-215205 (see page 2, etc.).

With this manipulator apparatus, an insertion hole is surgically incised in a body wall, for example, in the abdominal wall, an endoscope or treatment instrument is percutaneously inserted into the body cavity via this insertion hole, and examination or treatment is carried out inside the body cavity.

However, this conventional manipulator apparatus is of the type in which, after forming the insertion hole in the body wall or body cavity by incision, a trocar is placed in the insertion hole, and an endoscope or treatment instrument is inserted into the body wall or body cavity via the insertion hole, which is held open by the trocar. Therefore, it is necessary to carry out a surgical incision before the endoscope examination, and in addition, it is also necessary to use an instrument for the incision.

Furthermore, when carrying out examination of internal tissue or inside a comparatively narrow blood vessel of a small laboratory animal, such as a mouse or rat, since the object to be examined is small, it may be difficult to use the above-described method in which a trocar is positioned inside the insertion hole formed by incision.

### BRIEF SUMMARY OF THE INVENTION

The present invention has been conceived in light of the circumstances described above, and an object thereof is to provide an in-vivo examination apparatus that allows examination of a minute examination site inside a living organism with a simple configuration.

In order to realize the object described above, the present invention provides the following solutions.

According to a first aspect, the present invention provides an in-vivo examination apparatus including a light source; a flexible light-conveying member that transmits light from the light source to irradiate the light from an end face thereof onto an examination site and that receives return light returning from the examination site at the end face thereof to transmit the return light; a long thin insertion part in which the light-conveying member is disposed along the longitudinal direction thereof; and an optical detector that detects the return light from biological tissue, which is transmitted through the insertion part via the light-conveying member. The end face of the insertion part, where the end face of the light-conveying member is exposed, is configured so as to be cut at an angle with respect to the longitudinal direction to provide a pointed portion that can incise the biological tissue at the tip thereof.

According to this aspect of the invention, since the end face of the light-conveying member, which transmits light from the light source, is exposed at the end face of the insertion part, which has a shape formed by cutting it at an angle with respect to the longitudinal direction, it is possible to examine an examination site positioned in front and at an angle with respect to the longitudinal direction. In such a case, an incision can be made in the tissue with the pointed portion provided at the end face of the insertion part, and the end face of the light-conveying member can be positioned at an examination site located in the interior. Moreover, since the light-conveying member has flexibility, it can be freely flexed and inserted according to the shape of the incised tissue.

In this state, the light source is operated to irradiate the examination site from the end face with light from the light source, and return light returning from the examination site is received at the end face. The received return light is re-transmitted through the light-conveying member and is detected by the optical detector. In other words, according to this aspect of the invention, the end face of the light-conveying member can access an examination site positioned inside the tissue of the living organism to carry out examination without using a separate instrument for incision.

The aspect of the invention described above may also include an optical scanning unit that scans the light from the light source; and a focusing mechanism that focuses the light scanned by the optical scanning unit into the light-conveying member. The light-conveying member may be formed of an optical fiber bundle including a plurality of cores.

With this configuration, the light from the light source is scanned by operating the optical scanning unit, and the light is focused into each core of the optical fiber bundle constituting the light-conveying member by the operation of the focusing mechanism. The scanned light is emitted from the end face of the light-conveying member towards the examination site disposed opposite the end face, which allows examination of the examination site over a predetermined area to be carried out.

The aspect of the invention described above may also include a tip flexing mechanism for flexing the tip of the insertion part. The tip of the insertion part can be made to flex by operating the tip flexing mechanism, and it thus is possible to adjust the insertion direction to accurately locate the end face of the light-conveying member relative to the examination site.

In the aspect of the invention described above, the tip flexing mechanism may include an actuator formed of a shape-memory alloy, which is disposed at least along the longitudinal direction of the insertion part; and a temperature control unit that controls the temperature of the actuator. Since the actuator formed of a shape-memory alloy can be disposed in a small volume, the outer diameter of the insertion part can be reduced, which allows it to be inserted into a minute blood vessel or body cavity. By controlling the temperature of the actuator to a temperature determined in advance by operating the temperature control unit, the tip of the insertion part can be easily flexed and can thus be inserted in any direction.

In the aspect of the invention described above, the tip flexing mechanism may include a plurality of wires disposed along the longitudinal direction of the insertion part; and a tension control unit that individually applies tension to the plurality of wires. By individually applying tension to the wires by operating the tension control unit, the insertion part can be made to contract at the position in the circumferential direction where the wire to which tension is applied is disposed, which allows the insertion part to flex. Also, by changing the wire to which tension is applied, the direction in which the insertion part flexes can be changed.

In the aspect of the invention described above, a conduit may be formed in the longitudinal direction in at least one part of an outer face of the insertion part.

When the insertion part is inserted into a narrow body cavity, such as a blood vessel, the insertion part pushes against the narrow blood vessel to widen it while being inserted, and as a result, the blood vessel becomes obstructed and the blood flow is inhibited. With the above-described configuration, however, by means of the conduit formed in at least one part of the outer surface of the insertion part, blood can flow in the longitudinal direction along the conduit, which ensures the flow of blood. As a result, when carrying out in-vivo examination of a living organism, such as a small laboratory animal, it is possible to relieve the burden placed on the living organism.

In a preferable configuration of the aspect of the invention described above, the insertion part is attached to a casing accommodating at least the optical scanning unit and the focusing mechanism in such a manner that the insertion part can be rotated about the longitudinal axis thereof.

Because the pointed portion is formed by cutting the tip of the insertion part at an angle, the end face that irradiates light onto the living organism and that receives return light therefrom is also disposed at an angle with respect to the longitudinal direction of the insertion part. Therefore, by rotating the insertion part about the longitudinal axis thereof relative to the casing, it is possible to orient the end face in a direction suitable for examination of the examination site.

According to the present invention, the pointed portion provided at the tip of the insertion part can incise the tissue in the vicinity of the examination site, thus allowing examination of the examination site to be carried out. Therefore, an advantage is afforded in that it is possible to easily examine an examination site inside the tissue without the use of a separate instrument.

### BRIEF DESCRIPTION OF THE SEVERAL VIEWS OF THE DRAWINGS

FIG. 1. is an overall structural diagram showing an in-vivo examination apparatus according to an embodiment of the present invention.

FIG. 2 is a schematic longitudinal section showing the structure of an insertion part of the in-vivo examination apparatus in FIG. 1.

FIG. 3 is a diagram showing an actuator and a tension-adjusting device for flexing the end of the insertion part in FIG. 2.

FIG. 4 is a cross-sectional diagram showing the insertion part of the in-vivo examination apparatus in FIG. 1 when inserted into a blood vessel.

FIG. 5 is a schematic vertical cross-section showing a modification of a tip-flexing device in the insertion part of the in-vivo examination apparatus in FIG. 1.

FIG. 6 is a partially cut-away view showing a modification of the insertion part of the in-vivo examination apparatus in FIG. 1.

FIG. 7 is a diagram for explaining a seal used when inserting the insertion part in FIG. 6 into the tissue of a living organism.

### DETAILED DESCRIPTION OF THE INVENTION

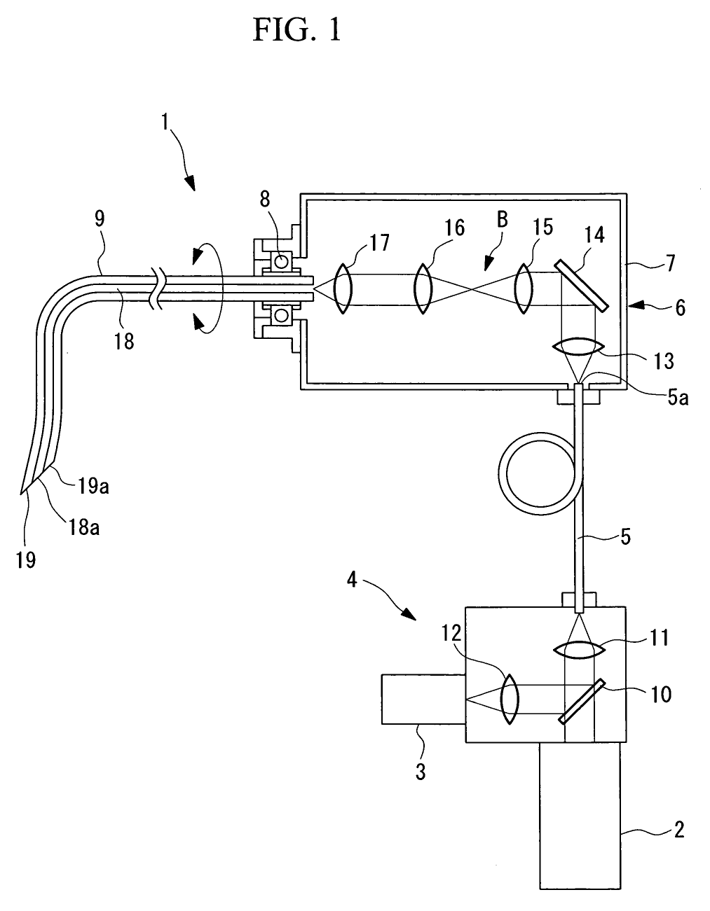

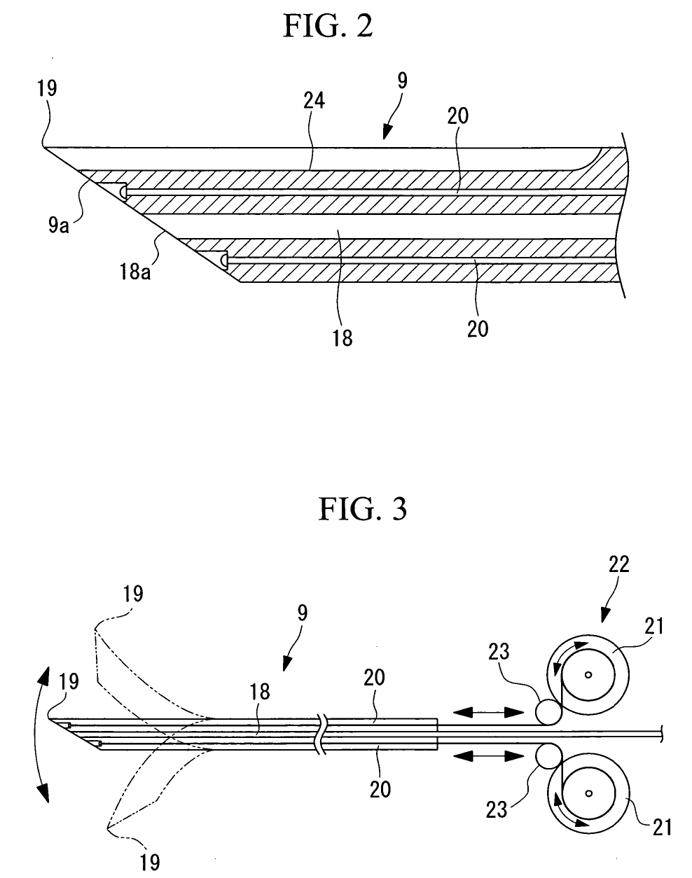

An in-vivo examination apparatus according to a first embodiment of the present invention will be described below with reference to FIGS. 1 and 2.

As shown in FIG. 1, an in-vivo examination apparatus 1 according to this embodiment includes an optical unit 4 having a laser light source 2 and an optical detector 3; an optical fiber 5 that transmits laser light from the laser light source 2 and fluorescence towards the optical detector 3; a measurement head 6 that two-dimensionally scans the laser light transmitted by the optical fiber 5; and an insertion part 9 that is supported so as to be rotatable about a longitudinal axis thereof by means of a bearing 8 in a casing 7 of the measurement head 6.

The optical unit 4 includes a dichroic mirror 10 that transmits excitation light from the light source 2 and that reflects fluorescence returning from the living organism; a focusing lens 11 that focuses the laser light onto the tip of the optical fiber 5; and a focusing lens 12 that focuses the fluorescence reflected by the dichroic mirror 10 onto the optical detector 3.

The measurement head 6 includes a collimator lens 13 for converting the laser light transmitted by the optical fiber 5 into a collimated beam; a galvano mirror (optical scanning unit) 14 that two-dimensionally scans the collimated beam; a pupil-projection lens 15 that forms an intermediate image of the beam scanned by the galvano mirror 14; an imaging lens 16 that gathers the light forming the intermediate image; and a focusing lens 17 that focuses the light gathered by the imaging lens 16 onto an end face of the insertion part 9.

The insertion part 9 includes an optical fiber bundle (light-conveying member) 18, having a plurality of optical fiber cores, along the entire length thereof on the central axis of a tube-shaped member formed of a flexible material. As shown in FIG. 1, the tip of the insertion part 9, as well as the optical fiber bundle 18, is formed so as to be cut at an angle with respect to the longitudinal direction. By doing so, a sharp pointed portion 19 is formed at the tip of the insertion part 9. When the pointed portion 19 is pressed against the tissue of a living organism, the tissue is incised, and at the same time, the tip of the insertion part 9 can be inserted into the interior thereof.

An end face 9a of the insertion part 9 where the pointed portion 19 is formed is oriented in a direction inclined with respect to the longitudinal direction, and an end face 18a of the optical fiber bundle 18 is exposed at this end face 9a. Accordingly, the end face of each optical fiber core constituting the optical fiber bundle 18 is in a conjugate positional relationship with respect to an intermediate image position B between the imaging lens 16 and the pupil projection lens 15 and the end face 5a of the optical fiber 5, and therefore, return light from the vicinity of the tissue with which the end face 18a of the optical fiber bundle 18 is in contact is selectively detected by the optical detector 3.

As shown in FIG. 2, a plurality of wires 20 are disposed at the tip of the insertion part 9 along the longitudinal direction. The plurality of wires 20 includes, for example, four wires disposed at intervals of 90° in the circumferential direction. One end of each of the wires 20 is fixed at the tip of the insertion part 9, and the other ends are connected to tension-adjusting devices 22 which wind up or let out the wires 20 by means of motors 21, as shown in FIG. 3. Reference numeral 23 in the figure represents tensioners.

With this arrangement, by operating the tension-adjusting device 22 connected to one of the wires 20 to increase the tension applied to that wire 20, it is possible to make the tip of the insertion part 9 flex in the radial direction in which that wire 20 is disposed. For example, as indicated by the chain line in FIG. 3, by applying tension to the wire 20 at the pointed portion 19 side, the insertion part 9 can be made to flex towards the pointed portion 19 side, and conversely, by relaxing the wire 20 on the pointed portion 19 side and applying tension to the wire 20 on the side away from the pointed portion 19, the insertion part 9 can be made to flex towards the opposite side from the pointed portion 19.

Also, in the in-vivo examination apparatus 1 according to this embodiment, a conduit 24 formed along the longitudinal direction is provided at the tip of the insertion part 9, as shown in FIG. 2. This conduit 24 is formed over a predetermined length from the tip of the insertion part 9.

The operation of the in-vivo examination apparatus 1 according to this embodiment, having such a configuration, will be described below.

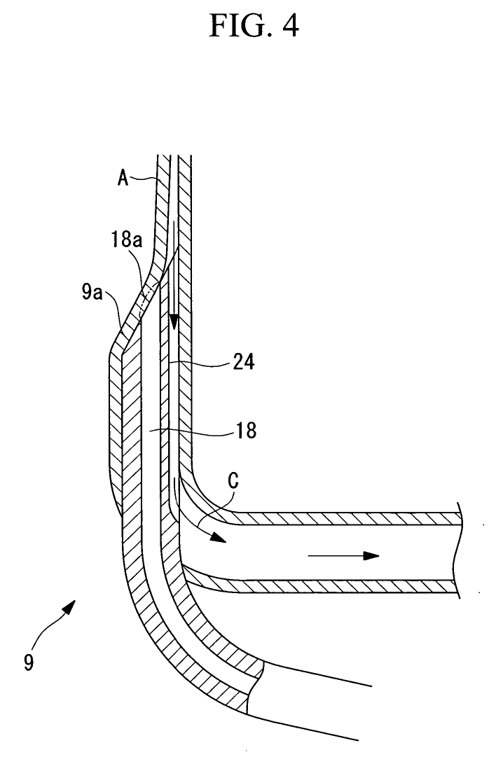

With the in-vivo examination apparatus according to this embodiment, when examining biological tissue, for example, the inner wall of a blood vessel A, as shown in FIG. 4, by pressing the pointed portion 19 of the insertion part 9 against the outer surface of the blood vessel A, the wall of the blood vessel A is cut by the pointed portion 19, which then passes therethrough, and the end face 9a of the insertion part 9 is positioned inside the blood vessel A. Since the pointed portion 19 is formed like a sharp edge by cutting the tip of the insertion part 9 at an angle with respect to the longitudinal direction, it can easily pass through the wall of the blood vessel A, and the end face 9a of the insertion part 9, in other words, the end face 18a of the optical fiber bundle 18, can be positioned inside the blood vessel A.

As shown in FIG. 4, the insertion part 9 is inserted inside the blood vessel A to a predetermined depth. More specifically, the insertion depth of the insertion part 9 is set such that the conduit 24 formed at the tip of the insertion part 9 is completely inserted into the blood vessel A. By doing so, as shown in FIG. 4, the blood flow C in the blood vessel A can continue to flow via the conduit 24 at the tip of the insertion part 9, even when the insertion part 9 pushes the blood vessel A apart when inserted into a blood vessel A that is narrower than the thickness of the insertion part 9. Therefore, when carrying out in-vivo examination of a living organism, such as a small laboratory animal or the like, it is possible to alleviate the burden placed on the living organism.

Furthermore, as shown in FIG. 4, the end face 18a of the optical fiber bundle 18, which is inserted into the blood vessel A, is placed in contact with the inner wall of the blood vessel A. By operating the optical unit 4 and the measurement head 6 in this state, the laser light transmitted via the optical fiber 5 from the laser light source 2 is two-dimensionally scanned by the optical scanning unit 14, is focused into the optical fiber bundle 18, and is emitted towards the inner wall of the blood vessel A from the end face 18a of the optical fiber bundle 18.

A fluorescent substance in the inner wall of the blood vessel A is excited by irradiation with the laser light and generates fluorescence. The generated fluorescence re-enters the optical fiber bundle 18 from the end face 18a of the optical fiber bundle 18, returns to the optical unit 4 via the focusing lens 17, the imaging lens 16, the pupil-projection lens 15, the optical scanning unit 14, the collimator lens 13, and the optical fiber 5, and is split off by the dichroic mirror 10 to be detected by the optical detector 3.

In this case, since the end face 18a of the optical fiber bundle 18 is disposed in conjugate positional relationship with the end face 5a of the optical fiber 5, the end face 5a of the optical fiber 5 functions as a confocal pinhole. Therefore, only fluorescence generated in the vicinity of the end face 18a of the optical fiber bundle 18 reaches the optical detector 3 to be detected.

Furthermore, in the case where no examination site is found on the inner wall of the blood vessel A with which the end face 18a of the optical fiber bundle 18 is in contact, or in the case where the examination site on the inner wall of the blood vessel A shifts, rather than changing the insertion depth of the insertion part 9, the end face 9a can be moved in the circumferential direction by rotating the insertion part 9 with respect to the casing 7 of the measurement head 6. By doing so, it is possible to place the end face 18a of the optical fiber bundle 18 in contact with a desired position on the inner wall of the blood vessel A to carry out examination.

With the in-vivo examination apparatus 1 according to this embodiment, by operating the tension-adjusting device 22 to change the tension applied to the wires 20, it is possible to flex the insertion part 9 so that the end face 9a thereof is oriented in a desired direction. Therefore, by flexing the insertion part 9 to change the orientation of the end face 9a and point the end face 18a of the optical fiber bundle 18 towards the front, it is possible to roughly examine the condition inside the blood vessel A at a region positioned in the forward insertion direction of the insertion part 9. Furthermore, flexing the insertion part 9 along the curvature of the blood vessel A allows it to proceed inside the blood vessel A.

Although the blood flow C is ensured by means of the conduit 24 provided in the outer surface of the insertion part 9, if the conduit 24 becomes blocked or if it becomes difficult to ensure a flow path due to the curvature of the blood vessel A, and so forth, it is possible to move the insertion part 9 like a snake to ensure a flow path for the blood by changing the direction of flexing of the insertion part 9 by operating the tension-adjusting device 22.

Therefore, with the in-vivo examination apparatus 1 according to this embodiment, it is possible to cut the tissue with the pointed portion 19 provided at the tip of the insertion part 9 to position the end face 18a of the optical fiber bundle 18 in the interior. Therefore, an advantage is provided in that it is possible to easily carry out examination without the need to use a separate device for incision. Also, since a flow path is ensured by the conduit 24 provided in the outer surface of the insertion part 9, it is possible to carry out examination while ensuring the flow of blood, even in biological tissue such as a narrow blood vessel A that is thinner than the outer diameter of the insertion part 9. Therefore, when carrying out in-vivo examination of a living organism, it is possible to alleviate the burden placed on the living organism, which allows examination to be performed for a long period of time.

Since the insertion part 9 can be flexed in a desired direction by the wires 20 provided in the insertion part 9 and by the operation of the tension-adjusting device 22, the end face 18a of the optical fiber bundle 18 can be made to proceed in a desired insertion direction inside the living organism. Therefore, the insertion part 9 can be inserted along a blood vessel A, body cavity, or the like that curves.

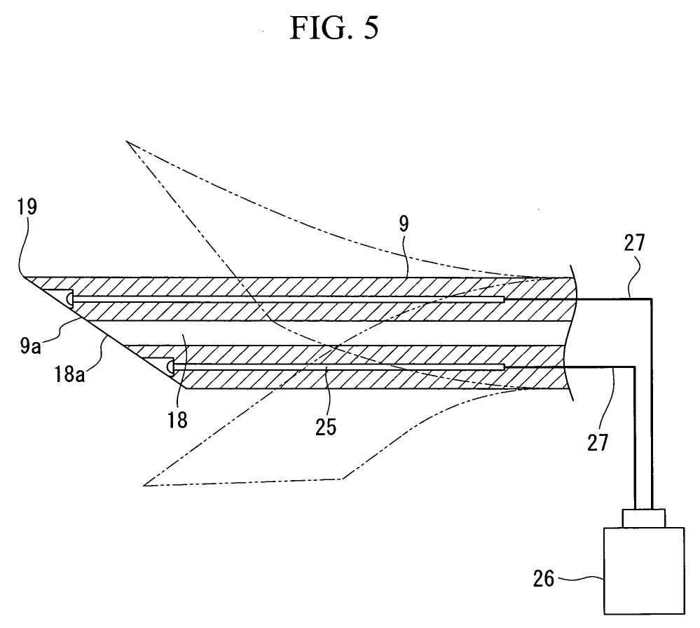

In the in-vivo examination apparatus 1 according to this embodiment, the wires 20, which are disposed in the longitudinal direction of the insertion part 9, and the tension-adjusting device 22, which applies tension to the wires 20, are used. Instead of this, however, as shown in FIG. 5, a wire-like actuator 25, formed of a shape-memory alloy and a heater, may be provided to extend over a predetermined length in the longitudinal direction in the vicinity of the tip of the insertion part 9, and temperature control of the actuator 25 may be carried out by supplying electrical power via a cable 27 connected to a temperature control unit 26.

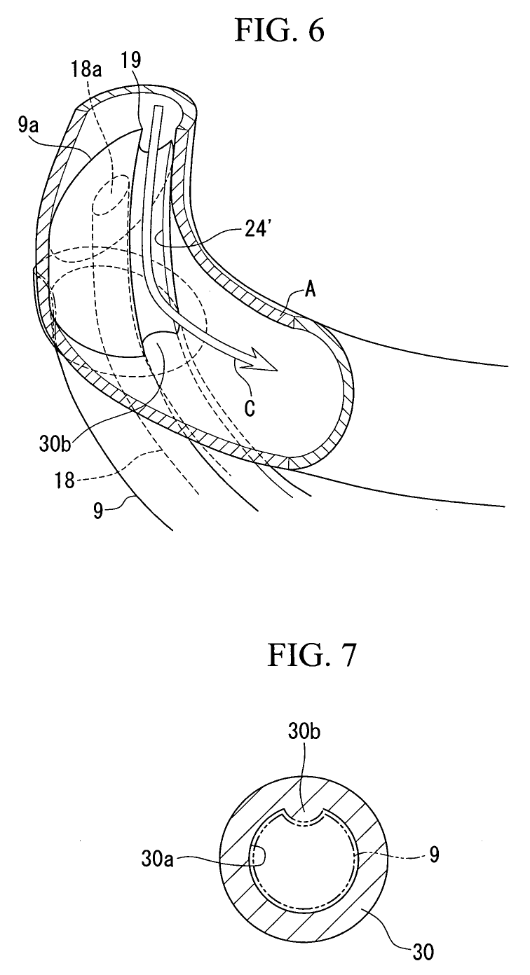

In the embodiment described above, the conduit 24, which is disposed in the outer surface of the insertion part 9, is partially provided over a length shorter than the insertion depth of the insertion part 9. Instead of this, however, a longer conduit 24′ may be formed. In such a case, blood can flow out via the conduit 24′ from a gap between the insertion part 9 and the wall of the blood vessel A, as shown in FIGS. 6 and 7, for example. Therefore, a relatively hard seal 30 having a hole 30a with the same shape as the cross-section of the insertion part 9 may be attached to the outer surface of the wall of the blood vessel A into which the insertion part 9 is inserted, and the outer shape of the insertion part 9 is made to be the same as the shape of the hole 30a so that it can be inserted therethrough. With this configuration, a protrusion 30b provided in the hole 30a of the seal 30 functions as a valve to seal off the conduit 24′ in the insertion part 9, and the amount of blood flowing outside is thus reduced. By doing so, an advantage is afforded in that it is possible to insert the insertion part 9 to a relatively deep position to carry out examination, without limiting the insertion depth of the insertion part 9.

What is claimed is:

1. An in-vivo examination apparatus comprising:
a light source;
a flexible light-conveying member that transmits light from the light source to irradiate the light from an end face thereof onto an examination site and that receives return light returning from the examination site at the end face thereof to transmit the return light;
a long thin insertion part in which the light-conveying member is disposed along the longitudinal direction thereof; and
an optical detector that detects the return light from examination site, which is transmitted through the insertion part via the light-conveying member;
wherein the end face of the insertion part, where the end face of the light-conveying member is exposed, is configured so as to be cut at an angle with respect to the longitudinal direction to provide a pointed portion that can incise examination site at the tip thereof.

2. An in-vivo examination apparatus according to claim 1, further comprising:
an optical scanning unit that scans the light from the light source; and
a focusing mechanism that focuses the light scanned by the optical scanning unit into the light-conveying member;
wherein the light-conveying member is formed of an optical fiber bundle including a plurality of cores.

3. An in-vivo examination apparatus according to claim 1, further comprising a tip flexing mechanism for flexing the tip of the insertion part.

4. An in-vivo examination apparatus according to claim 3, wherein the tip flexing mechanism includes:
an actuator formed of a shape-memory alloy, which is disposed at least along the longitudinal direction of the insertion part; and
a temperature control unit that controls the temperature of the actuator.

5. An in-vivo examination apparatus according to claim 3, wherein the tip flexing mechanism includes:
a plurality of wires disposed along the longitudinal direction of the insertion part; and
a tension control unit that individually applies tension to the plurality of wires.

6. An in-vivo examination apparatus according to claim 1, wherein a conduit is formed in the longitudinal direction in at least one part of an outer face of the insertion part.

7. An in-vivo examination apparatus according to claim 2, wherein the insertion part is attached to a casing accommodating at least the optical scanning unit and the focusing mechanism in such a manner that the insertion part can be rotated about the longitudinal axis thereof.
